(19) United States
(12) Patent Application Publication
(10) Pub. No.: US 2022/0245389 A1
(43) Pub. Date: Aug. 4, 2022

(54) EMOTION RECOGNITION METHOD AND SYSTEM BASED ON HEART-EXPRESSION SYNCHRONIZATION

(71) Applicant: SANGMYUNG UNIVERSITY INDUSTRY-ACADEMY COOPERATION FOUNDATION, Seoul (KR)

(72) Inventors: Hyun Woo LEE, Goyang-si (KR); A Young CHO, Goyang-si (KR); Min Cheol WHANG, Goyang-si (KR)

(73) Assignee: SANGMYUNG UNIVERSITY INDUSTRY-ACADEMY COOPERATION FOUNDATION, Seoul (KR)

(21) Appl. No.: 17/393,179

(22) Filed: Aug. 3, 2021

(30) Foreign Application Priority Data
Feb. 2, 2021 (KR) 10-2021-0014974

(51) Int. Cl. G06K 9/03; G06K 9/00; G06K 9/62

(52) U.S. Cl. CPC G06K 9/03; G06K 9/00315; G06K 9/629; G06K 9/00281; G06K 9/0061

(57) ABSTRACT

The present disclosure provides a method and system for emotional evaluation based on heart-face movement synchronization. The evaluation method extracts a subject's facial image and heart information, extracts micro-movement data of an action unit (AU) defined in a face region from the facial image, extracts heart-evoked micro-movement (HEMM) data from micro-movement data in synchronization with a heartbeat characteristic signal obtained from heart information, extracts one or more characteristic parameters for the AU movement from the HEMM, evaluates the emotion of the subject using the characteristic parameter.

### CROSS-REFERENCE TO RELATED APPLICATION

This application is based on and claims priority under 35 U.S.C. § 119 to Korean Patent Application No. 10-2021-0014974, filed on Feb. 2, 2021, in the Korean Intellectual Property Office, the disclosure of which is incorporated by reference herein in its entirety.

### BACKGROUND

### 1. Field

The present disclosure relates to a method and system for emotion recognition based on synchronization of heart and facial expressions.

### 2. Description of the Related Art

A research on emotional computing to improve the user's experience for human-computer interaction (HCl) has been continuously conducted. Recently, user experience has been recognized as an important element of products beyond the improvement of functions. In order to improve the user experience, it is necessary to recognize a user's emotions in daily life. When a product recognizes the user's emotions and responds appropriately to the recognized motions, it may increase the value of the product. In emotional computing, emotions are recognized from behaviors and physiological responses.

Facial expressions are representative emotional behavioral responses that may be recognized by a camera in everyday life. These facial expressions were classified into various emotions and studied, and it was confirmed that the same emotion accompanies similar expressions and gestures regardless of race. A facial action coding system (FACS) defines an action unit (AU) as the individual components of muscle movements that exhibit various facial expressions. The facial expressions are recognized by the motion classification of AU and their emotional state is evaluated. This emotional state may be measured intuitively and conveniently in a non-contact method using a camera. However, it is difficult to recognize human internal emotions from facial expressions. For example, a person may consciously smile on the outside while being angry on the inside. At this point, a computer may mistakenly assume that the person is happy.

In the conventional emotion evaluation method, emotion is recognized by independently considering a heart reaction and a facial expression. That is, conventionally, the evaluation of an implicit emotion using a cardiac response and the evaluation of an explicit emotion through the change of the facial expression are separately performed. Although it is possible to individually judge implicit and explicit emotions by these methods, it may fail to evaluate true emotions in which both implicit and explicit emotions are complexly reflected, and studies are needed to correct these evaluation errors.

### SUMMARY

The present disclosure provides a method for more accurately recognizing or evaluating an emotional state and an apparatus for applying the same.

The present disclosure provides a method and apparatus capable of accurately recognizing or evaluating internal emotions through synchronization of heart information and facial expressions.

Additional aspects will be set forth in part in the description which follows and, in part, will be apparent from the description, or may be learned by practice of the presented embodiments of the disclosure.

An emotion evaluation method according to one or more embodiments may include

taking a facial image of a subject using a camera;

extracting heart information of the subject using a heart information sensor;

extracting micro-movement information of one or more action units (AUs) defined in a face region from the face image by an image processor;

extracting heart-evoked micro-movement (HEMM) data from the micro-motion information in synchronization with a heartbeat characteristic signal obtained from the heart information by an information processor;

extracting one or more characteristic parameters for AU motion from the HEMM data; and

evaluating the emotion of the subject using the characteristic parameter by an emotion evaluator.

According to one or more embodiments, the heartbeat characteristic signal may be obtained from PPG or ECG information.

According to one or more embodiments, the HEMM data is extracted from a peak power generation point in the heartbeat characteristic signal for a predetermined time (t), and according to an embodiment, the predetermined time (t) may be set to 0.5 seconds.

According to one or more embodiments, at least one of Mean, standard deviation (SD), positive power peak (PPP), positive peak time (PPT), negative peak power (NPP), and negative peak time (NPT) from the HEMM data may be calculated as the characteristic parameter.

According to one or more embodiments, the AU may include at least one of inner brow raise AU1, outer brow raise AU2, upper lid raise AUS, cheek raise AU6, lids tight AU7, nose wrinkle AU9, lip corner puller AU12, lip corner depressor AU15, lower lip depress AU16, lip stretch AU20, lip funneler AU23, and jaw drop AU26.

According to one or more embodiments, the AU may evaluate at least one of six basic emotions such as happiness HA, sadness SA, surprise SU, anger AN, disgust DI, and fear FE using the characteristic parameter.

An emotion evaluation system according to one or more embodiments may include

a facial imaging camera configured to photograph a subject's face;

a heart information extractor configured to extract heart information of the subject;

an information processor configured to process an image from the facial imaging camera to extract micro-movement data of an AU defined on the subject's face, wherein micro-movement (HEMM) data of the AU is extracted based on the heart information of the subject, and a characteristic parameter for the movement of the AU is extracted from the micro-movement data; and

an emotion evaluator configured to evaluate an emotion displayed on the subject's face using the characteristic parameter.

According to one or more embodiments, the information processor may extract the HEMM data from a peak power generation point in the heartbeat characteristic signal for a predetermined time (t).

According to one or more embodiments, the information processor may calculate at least one of Mean, standard deviation (SD), positive power peak (PPP), positive peak time (PPT), negative peak power (NPP), and negative peak time (NPT) from the HEMM data as the characteristic parameter.

According to one or more embodiments, the AU may include at least one of inner brow raise AU1, outer brow raise AU2, upper lid raise AUS, cheek raise AU6, lids tight AU7, nose wrinkle AU9, lip corner puller AU12, lip corner depressor AU15, lower lip depress AU16, lip stretch AU20, lip funneler AU23, and jaw drop AU26.

According to one or more embodiments, the AU may evaluate at least one of six basic emotions such as happiness HA, sadness SA, surprise SU, anger AN, disgust DI, and fear FE using the characteristic parameter.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows an anatomically interconnected structure through a brain.

FIG. 2 is a block diagram illustrating an emotion recognition method according to the present disclosure.

FIG. 3 is a block diagram explaining in detail the evaluation of heart-evoked micro-movement (HEMM).

FIG. 4 shows an outline of a main part of a face detected by Ekman's FACS.

FIG. 5 shows an arrangement of landmarks for various parts of a face located on a contour line according to the definition of FACS.

FIG. 6 illustrates 13 facial muscle AUs with landmarks defined in the major facial regions.

FIG. 7 illustrates facial muscle AUs in six emotions.

FIG. 8 shows an extraction process of HEMM data according to the present disclosure.

FIG. 9 illustrates a pattern of mean, which is one of HEMM characteristic parameters according to the present disclosure.

FIG. 10 shows an SD pattern, which is one of HEMM characteristic parameters, according to the present disclosure.

FIG. 11 shows patterns of PPP and NPP, which are HEMM characteristic parameters, according to the present disclosure.

FIG. 12 shows a pattern of PPT and NPT, which are HEMM characteristic parameters according to the present disclosure.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, preferred embodiments of the present invention concept will be described in detail with reference to the accompanying drawings. However, embodiments of the inventive concept may be modified in various other forms, and the scope of the inventive concept should not be construed as being limited by the embodiments described below. The embodiments of the inventive concept are preferably interpreted as being provided to more completely explain the inventive concept to those of ordinary skill in the art. The same symbols refer to the same elements from time to time. Furthermore, various elements and regions in the drawings are schematically drawn. Therefore, the inventive concept is not limited by the relative size or spacing drawn in the accompanying drawings.

Terms such as first, second, etc. may be used to describe various elements, but the elements are not limited by the terms. The above terms are used only for the purpose of distinguishing one element from another. For example, without departing from the scope of the inventive concept, the first element may be referred to as the second element, and conversely, the second element may be referred to as the first element.

The terms used in the present application are only used to describe specific embodiments, and are not intended to limit the inventive concept. The singular expression includes the plural expression unless the context clearly dictates otherwise. In the present application, it should be understood that expressions such as “include” or “have” are intended to designate that a feature, number, step, operation, element, part, or combination thereof described in the specification exists, but does not preclude the possibility of presence or addition of one or more other features or numbers, operations, components, parts, or combinations thereof.

Unless defined otherwise, all terms used herein have the same meaning as commonly understood by one of ordinary skill in the art to which the inventive concept belongs, including technical and scientific terms. Also, it will be understood that commonly used terms as defined in advance should be construed to have a meaning consistent with what they mean in the context of the relevant technology, and not to be construed in an overly formal sense unless explicitly defined herein.

In cases where certain embodiments may be implemented differently, a specific process sequence may be performed differently from the described sequence. For example, two processes described in succession may be performed substantially simultaneously, or may be performed in a sequence opposite to the described sequence.

Hereinafter, an emotion recognition system based on heart-expression synchronization and a method thereof according to one or more embodiments will be described in detail.

A cardiac response is a representative physiological response for recognizing emotions in daily life. In particular, a heart rate is an important biosignal in emotional computing due to the monitoring of the autonomic nervous system (ANS). The heart rate repeatedly rises or falls in response to breathing, blood pressure, and emotions. Heart rate variability (HRV) is an analysis of subtle changes in heart rate between one cardiac cycle and the next. In particular, short-term HRV of 3 minutes to 5 minutes is affected by ANS activity by emotional changes. Because a heart response is not consciously controlled, the heart response reflects the true emotion that is implicitly felt. Accordingly, the present disclosure provides a method and system for more accurately recognizing emotion by integrating the explicit and implicit responses considering both facial expressions and cardiac responses.

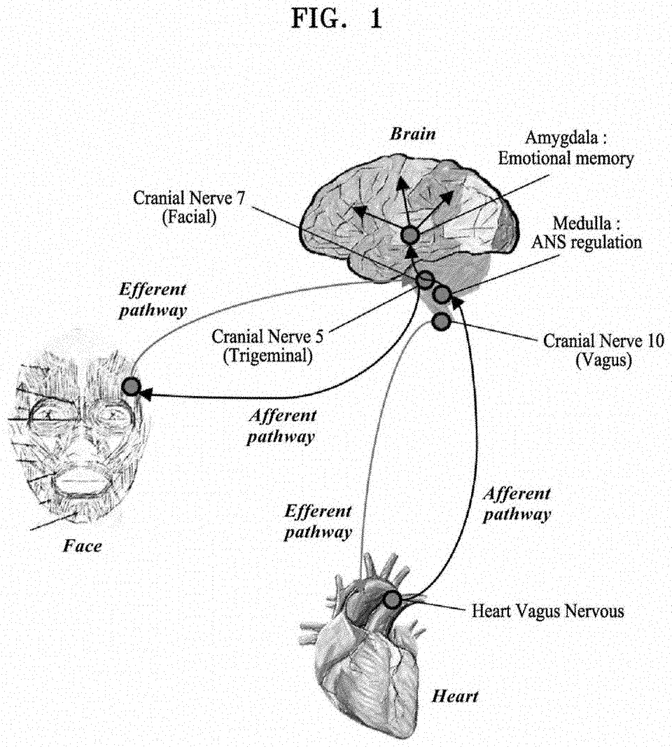

The heart and facial muscles are anatomically interconnected via the brain as shown in FIG. 1. A facial expression is controlled by the movement or position of muscles micheleunder the skin of a face.

Muscles related to the facial expression are largely classified into 14 muscles (e.g., frontal muscles, corrugator, etc.) The 14 muscles are controlled by two cranial nerves in the brain. A first cranial nerve 5 is called a trigeminal nerve and extends from the pons. A main function of the cranial nerve 5 is to receive sensations from the face and to control four muscles of mastication. A second cranial nerve 7 is called the facial nerve, also extends from the pons, and stimulates the motor function of the facial expression muscles. In addition, the cranial nerve 7 innervates two small muscles known as the stapedius muscles and a posterior belly of digastric muscle. Parasympathetic fibers of the head and neck ganglia are also supplied by the trigeminal and facial nerves.

A heart also interacts with the brain by a cranial nerve 10 called the vagus nerve. The cranial nerve 10 transmits information of internal organs from major organs such as the heart to the brain through afferent pathways. The information of internal organs is integrated into the brainstem and then passed on to the amygdala, which manages emotional memory. In addition, the information of internal organs is transmitted to a prefrontal cortex through a hypothalamus and thalamus of a midbrain. The prefrontal cortex affects mental activities such as attention and cognition. Finally, brain information is transmitted from the vagus nerve to the heart via efferent pathways. Emotions in the brain influence the parasympathetic and sympathetic responses of the heart. Based on this anatomical connection, it may be seen that information generated from the heart is transmitted to the face through the brain.

Therefore, a real facial expression accompanied by emotion indicates heart-face synchronization, whereas a fake facial expression without emotion indicates heart-face asynchrony. A more accurate inner emotion may be extracted using such synchronization and desynchronization.

An emotional evaluation system according to one or more embodiments may include a facial image camera for photographing a subject's face and a heart information extractor for extracting heart information of the subject, for example, an ECG or PPG device, an image processor for extracting micro-motion information for processing a facial image, and an information processor for processing the micro-motion information and heart information.

The information is processed and analyzed by a computer-based device having a processor for information processing and an evaluator that performs judgment of emotion using the processed information. The processor processes the image from the facial imaging camera to extract micro-motion data of the action unit (AU) defined on the subject's face, wherein the processor extracts micro-motion (HEMM) data of the AU based on the subject's heart information and extracts characteristic parameters for the movement of the AU from the micro-movement data. The evaluator evaluates the emotion displayed on the subject's face by using the characteristic parameter.

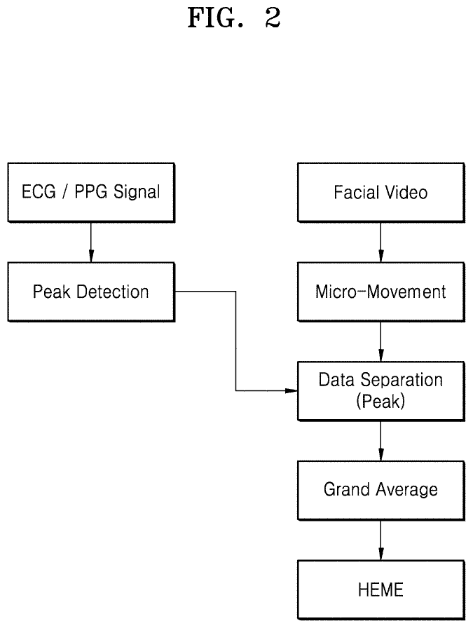

The emotion recognition method according to the present disclosure is as shown in FIG. 2 and includes the following three processes.

Micro-movement (MM) described below means the movement of one or more AUs consisting of landmarks defined on the face. In the present disclosure, the actual emotion shown in the facial expression is evaluated by evaluating the movement of these AUs.

1) MM Extraction

In this step, micro-motion (MM) of the AU is extracted by FACS while photographing the subject's face.

2) HEMM (Heart Evoked Micro-Movement) Analysis

In this step, detection of ECG/PPG signals of the subject is performed simultaneously with facial imaging for MM extraction. HEMM data is a heartbeat characteristic signal extracted from ECG or PPG in raw MM, for example, a signal of a periodic constant section extracted from a peak origin signal for a predetermined period.

3) Emotion Recognition

The emotion recognition is performed using the HEMM extracted based on a peak signal of ECG/PPG. In this process, a grand average is obtained from the signals of a periodic constant section, HEMM data is calculated therefrom, and the emotion is evaluated by the HEMM data. The HEMM data specifies the emotion shown in the subject's facial expression by judging the movement of a specific facial muscle AU corresponding to a specific emotion.

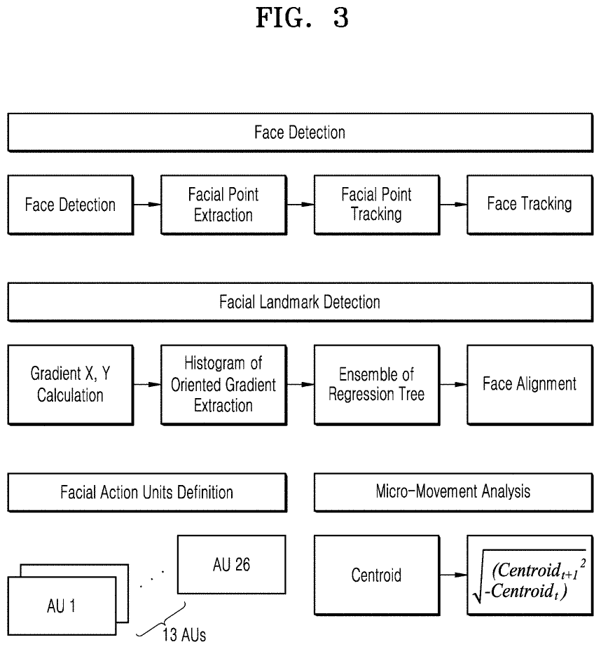

FIG. 3 is a block diagram specifically explaining the evaluation of the HEMM.

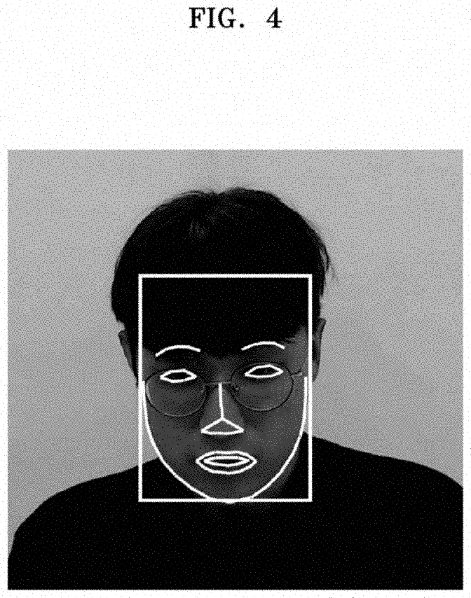

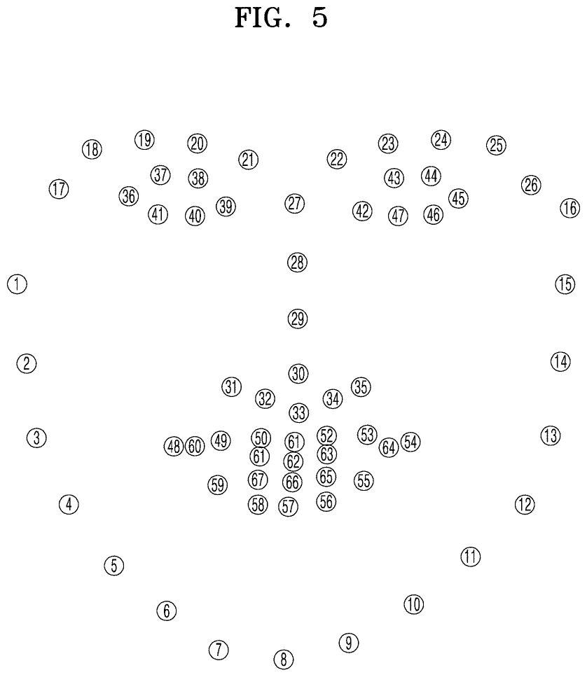

FIG. 4 shows outlines of the main parts of the face detected by Ekman's FACS, and FIG. 5 shows the arrangement of landmarks for various parts of the face located on a contour line according to the definition of FACS.

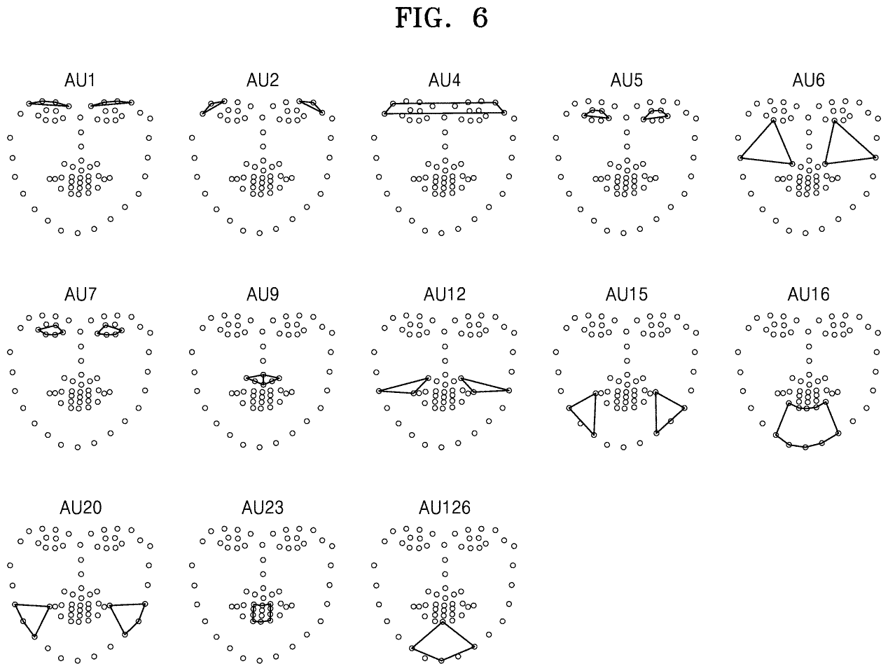

FIG. 6 illustrates 13 facial muscle AUs by landmarks defined on major facial regions.

As shown in FIG. 6, landmarks are grouped by a yellow line, and AUs mean an area within a plurality of landmarks connected by the yellow line. Table 1 below describes the AU, which defines a movement of the facial part that causes a change in facial expression, and the corresponding landmark and muscle movement related thereto.

TABLE 1

| AU | Description | Facial Landmarks |
|---|---|---|
| AU 1 | Inner Brow Raise | 18, 19, 20, 21 (Left) |
| | | \| 22, 23, 24, 25(Right) |
| AU 2 | Outer Brow Raise | 17, 18, 19 (Left) |
| | | \| 24, 25, 26 (Right) |
| AU 4 | Brow Lowerer | 17, 18, 25, 26 |
| AU 5 | Upper Lid Raise | 36, 37, 38, 39 (Left) |
| | | \| 42, 43, 44, 45 (Right) |
| AU 6 | Cheek Raise | 41, 2, 31 (Left) |
| | | \| 46, 14, 35(Right) |
| AU 7 | Lids Tight | 36, 41, 39, 38 (Left) |
| | | \| 42, 46, 45, 43 (Right) |
| AU 9 | Nose Wrinkle | 30, 31, 33 (Left) |
| | | \| 30, 33, 35 (Right) |
| AU 12 | Lip Corner Puller | 3, 48, 31 (Left) |
| | | \| 13, 54, 35 (Right) |
| AU 15 | Lip Corner Depressor | 4, 6, 48 (Left) |
| | | \| 12, 54, 10 (Right) |
| AU 16 | Lower Lip Depress | 59, 6, 7, 8, 9, 10, 55, 56, 57, 58 |
| AU 20 | Lip Stretch | 3, 4, 5, 48 (Left) |
| | | \| 11, 12, 13, 54 (Right) |
| AU 23 | Lip Funneler | 50, 58, 56, 52 |
| AU 26 | Jaw Drop | 6, 8, 10, 57 |

Hereinafter, the process shown in FIG. 3 will be described in more detail.

I. Extraction of Facial Micro-Movement

An extraction of micro-movement follows the process of extracting micro-movement of a specific AU as shown in FIG. 3.

First, a face region or point is extracted through face detection and tracking. An extraction of the face region may be performed by a conventional face detection algorithm such as a Viola-Jones algorithm, a histogram of oriented gradients (HOG) algorithm, or deep learning. As shown in FIG. 5, the face region extracted in the above process and 68 landmarks extracted from the face region are tracked, and these landmarks may be detected by a typical face landmark detection algorithm such as DLIB or deep learning.

FIG. 6 shows 13 AUs based on Ekman's FACS based on face landmarks, and shows the results of mapping the combinations of AUs accompanying 6 emotions based on FACS.

TABLE 2

| Emotion | AU |
|---|---|
| Happiness | AU 6, AU 12 |
| Sadness | AU 1, AU 4, AU 15 |
| Surprise | AU 1, AU 2, AU 5, AU 26 |
| Anger | AU 4, AU 5, AU 7, AU 23 |
| Disgust | AU 9, AU 15, AU 16 |
| Fear | AU 1, AU 2, AU 4, AU 5, AU 7, AU 20, AU 26 |

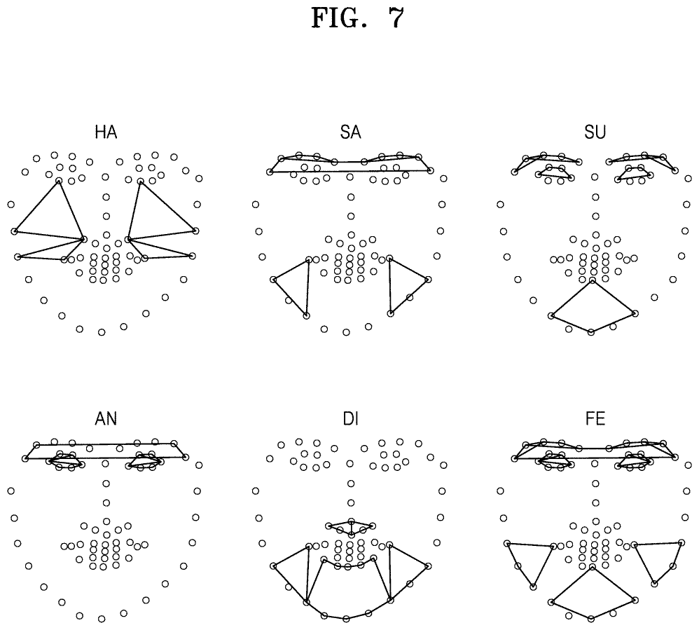

As defined in Table 2 above, each face muscle AU is associated with six basic emotions: happiness (HA), sadness (SA), surprise (SU), anger (AN), disgust (DI), and fear (FE). FIG. 7 shows the facial muscle AUs in the six emotions.

For each facial muscle, the area Axy of the AU and the x, y coordinates Cx, Cy of the centroid of the AU are calculated, based on a polygonal structure from the x, y coordinates of the face landmarks constituting the facial muscles.

$$\begin{matrix}{A}_{xy} = \frac{1}{2} ( \vert \begin{matrix}{x}_{1} & {x}_{2} \\ {y}_{1} & {y}_{2}\end{matrix} \vert + \vert \begin{matrix}{x}_{2} & {x}_{3} \\ {y}_{2} & {y}_{3}\end{matrix} \vert + ⋯ + \vert \begin{matrix}{x}_{n} & {x}_{1} \\ {y}_{n} & {y}_{1}\end{matrix} \vert ) = \frac{1}{2} ( {x}_{1} {y}_{2} - {x}_{2} {y}_{1} + {x}_{2} {y}_{3} - {x}_{3} {y}_{2} + ⋯ + {x}_{n - 1} {y}_{n} - {x}_{n} {y}_{n - 1} + {x}_{n} {y}_{1} - {x}_{1} {y}_{n} ) & [ Equation 1 ] \\ {C}_{x} = \frac{1}{6 Area} {\sum }_{i = 0}^{n - 1} ( {x}_{i} + {x}_{i + 1} ) ( {x}_{i} {y}_{i + 1} - {x}_{i + 1} {y}_{i} ) & [ Equation 2 ] \\ {C}_{y} = \frac{1}{6 Area} {\sum }_{i = 0}^{n - 1} ( {y}_{i} + {y}_{i + 1} ) ( {x}_{i} {y}_{i + 1} - {x}_{i + 1} {y}_{i} ) & [ Equation 3 ]\end{matrix}$$

In Equations 1, 2, and 3 above, x is the horizontal coordinate of the face landmark, y is the vertical coordinate of the face landmark, n is the number of landmarks constituting the AU, and Axy is the area, Cx is the x-coordinate of the centroid of the AU, and Cy is the y-coordinate of the centroid of the AU. Finally, from the difference in the center coordinates of the facial muscles between adjacent frames, the movement distance of a specific AU or the distance moved by the specific AU, that is, V_{MM}, which is a micro-movement value, is extracted as a raw signal as follows.

[Equation 4]

V_{MM}=\sqrt{(C_{x,t+1}−C_{x,t})²+(C_{y,t+1}−C_{y,t})²}

In Equation 4 above, V_{MM} is the value of micro-movement of the AU, Cx-t is the x-coordinate of the center of the AU for a frame for a given period of time t, and Cy-t is the y-coordinate of the center of the AU for a frame at a given time t.

II. Extraction of Heart Evoked Micro-Movement (HEMM)

The HEMM represents the characteristics of heart-expression synchronization analyzed from cardiac signals and micro-movements of AU.

In the present disclosure, information sent from the heart to the brain through the afferent path is transmitted from the brain to the face through the efferent path, thereby extracting the value of the heart evoked micro-movement at the visceral level. For this, V_{MM} is extracted based on the cardiac signal cycle. That is, in extracting the V_{MM} in the present disclosure, the time of occurrence of a specific signal obtained from the heart is applied as an extraction timing signal of the MM value, that is, a synchronization signal for extraction of V_{MM}, and the heart signal-based MM signal, that is, the HEMM, is continuously extracted from the synchronization signal for a predetermined period. According to a specific embodiment, a peak (timing) of a heart-related signal extracted from ECG/PPG may be used as the synchronization signal for MM extraction.

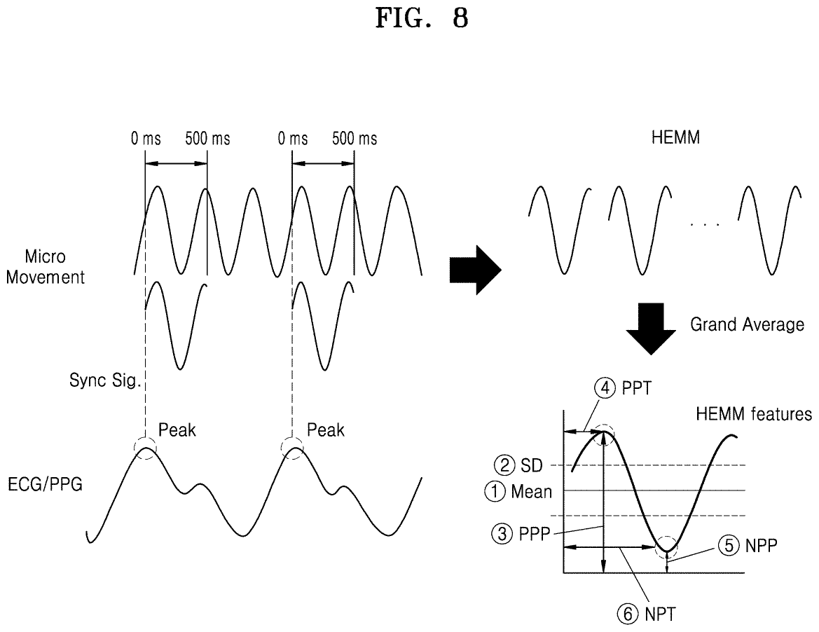

The extraction process of HEMM is as shown in FIG. 8 First, a peak is detected as a sync signal from a cardiac signal ECG/PPG. Because the MM of the corresponding AU according to a specific emotion occurs within a predetermined time t, 0.5 seconds according to the present disclosure, the MM of the AU may be extracted by dividing the time t by a length unit of 0.5 seconds from the peak (synchronization signal) of the heart signal as a reference time. The HEMM obtained through this process was finally extracted as the grand average of MM separated for each facial muscle AU. Six characteristic parameters such as Mean, standard deviation (SD), positive power peak (PPP), positive power time (PPT), negative peak power (NPP), and negative peak time (NPT) are calculated from the finally extracted HEMM. These characteristic parameters have characteristics as shown in Table 3 below.

TABLE 3

| Feature | Description |
|---|---|
| Mean | Strength of afferent pathway from heart to face |
| SD | Reactivity of face for afferent information from heart |
| PPP | Strength of positive peak in afferent information from heart |
| | to face |
| PPT | Latency time of positive peak in afferent information from |
| | heart to face |
| NPP | Strength of negative peak in afferent information from heart |
| | to face |
| NPT | Latency time of negative peak in afferent information from |
| | heart to face |

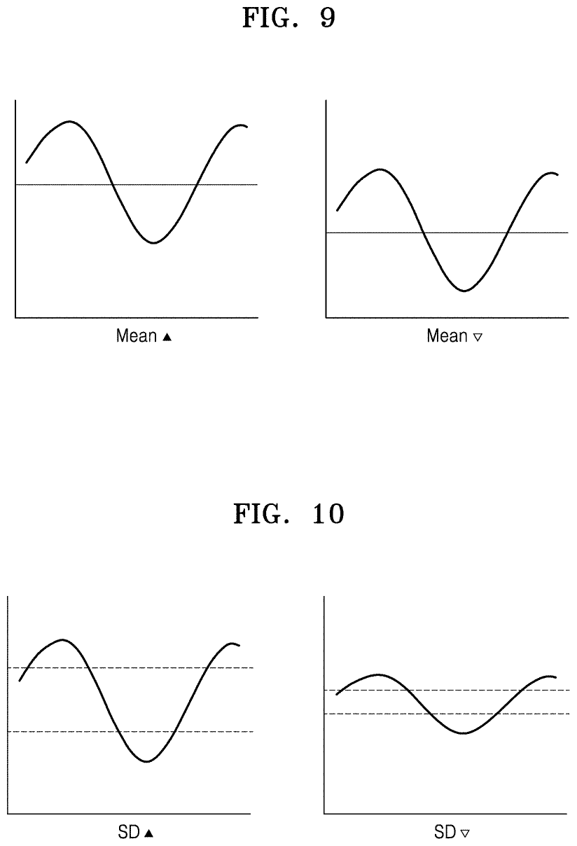

(1) The parameter Mean, grand average is the average power value of HEMM′ and is extracted as follows. The Mean represents the intensity of MM with respect to the cardiac cycle, and thus is related to the intensity of afferent information from the heart to the face. The Mean is an indicator of ANS activation, and the Mean pattern of the HEMM is as shown in FIG. 9.

$$\begin{matrix}{l}_{ength = fps} \times 0.5 & [ Equation 5 ] \\ Mean = \frac{1}{{l}_{ength}} \times {\sum }_{i = 1}^{{l}_{ength}} {HEMM}_{i} & [ Equation 6 ]\end{matrix}$$

In Equations 5 and 6 above, length is the length of the HEMM, and fps is the number of frames per second of the face image.

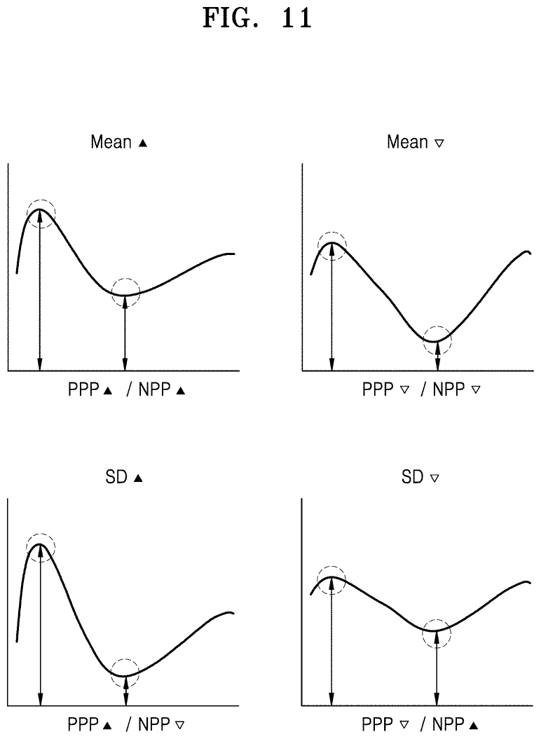

(2) The parameter SD is the standard deviation of HEMM and is extracted as follows. Because the parameter SD represents the stability of MM with respect to the cardiac cycle, the parameter SD is related to the reactivity of the face to the afferent information transmitted from the heart. The parameter SD is an index for the MM responsiveness based on ANS activation, and the SD pattern of the HEME is as shown in FIG. 10.

$$SD = \sqrt{\frac{{\sum }_{i = 1}^{length} {( {HEMM}_{i} - Mean )}^{2}}{length - 1}} \qquad [ Equation 7 ]$$

(3) The parameter PPP is the maximum value of the power value of the positive peak in the HEMM.

[Equation 8]

PPP=max(HEMM)

The parameter PPP represents the intensity (strength) of the strongest MM for the cardiac cycle, which is related to the strength of the strongest afferent information from the heart to the face.

(4) The parameter PPT is the delay time of the HEMM's positive peak and is extracted as follows. The parameter PPT is related to the delay time until the strongest afferent information from the heart to the face occurs.

$$PPT = argmax ( HEMM ) \times \frac{1}{fps} \qquad [ Equation 9 ]$$

(5) The parameter NPP is the power value of the negative peak of HEME and is extracted as follows. The parameter NPP represents the strength of the weakest micro-expression for the cardiac cycle. The parameter NPP is related to the strength of the weakest afferent information from the heart to the face.

$$NPT = argmin ( HEMM ) \times \frac{1}{fps} \qquad [ Equation 10 ]$$

(6) The parameter NPT is the delay time of the negative peak of HEME and is extracted as follows. The parameter NPT is related to the delay time until the weakest afferent information from the heart to the face occurs.

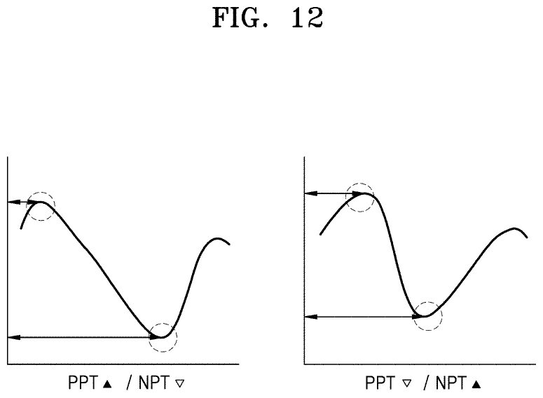

In HEMM analysis, Mean and SD may be evaluated separately as shown in FIG. 11 by the effects of PPP and NPP. Also, the duration time of interconnection between the heart and the face may be evaluated by the patterns of PTT and NPT as shown in FIG. 12.

III. Emotion Recognition or Evaluation

By applying the above parameters, the subject's true emotion or sensibility are evaluated. According to the present disclosure, evaluation of (1) comfort-awakening, (2) discomfort-relaxation, (3) discomfort-awakening, (4) comfort-relaxation belonging to a two-dimensional emotional model using HEMM features (parameters) can be performed. Hereinafter, L or R added to the rear end of the AU indicates the left part or the right part of the AU, and the increase or decrease of the parameters described below is a result of comparing data when intentionally expressing false emotions with a face (FAKE) and when expressing real inner emotions with a face (TRUE).

A. Comfort-Awakening (Happiness, HA)

With respect to comfort-awakening (HA), facial muscle AUs to be activated include AU4, AU9L, AU9R, AU23.

In the comfort-awakening state, autonomic and sympathetic nerves are activated and parasympathetic nerves are deactivated. In the comfort-awakening state, the parameter Mean increases because the amount of information sent from the heart to the face increases, and also the parameter SD increases because the reactivity increases.

In addition, as the temporal persistence of the interconnection between the face and the heart increases, PPT decreases and NPT increases.

B. Discomfort-Relaxation (Sadness, SA)

Regarding discomfort-relaxation (SA), right brain-controlled facial muscle AUs associated with negative emotions include AU5L: AU7L, AU9L, AU15L, and AU20L.

In the discomfort-relaxation state, the heart rate decreases and the parasympathetic nerves are activated. In addition, because the amount of information sent from the heart to the face increases, the parameter Mean increases, and the change in reactivity does not appear significantly and remains neutral.

In addition, because the temporal persistence of the interconnection between the face and the heart decreases, PPT increases and NPT decreases.

C. Discomfort-Awakening (Anger, AN)

Regarding discomfort-awakening (AN), the left-brain-controlled right facial muscle associated with negative emotion is inactivated, and AUs belonging to this include AU5R: AU6R, and AU7R.

In the discomfort-awakening state, the heart rate decreases and the sympathetic nervous system is deactivated. In addition, the response of the face to the information delivered from the heart decreased, so the parameter SD decreased, and the PPP, which had no significant change in other emotions, decreased.

D. Comfort-Relaxation (satisfaction)

Regarding comfort-relaxation emotions, facial muscle AUs include AU2L, AU7R, AU9L, and AU15R.

In this state, the heart rate slows and the parasympathetic nerves are activated. At this time, the change of parameters Mean, SD, PPP, and NPP was small, and there was a tendency that the NPT pattern disappeared. In addition, the persistence of the upper muscles decreased and the persistence of the lower muscles increased. This seems to be because the emotional intensity is weak in the comfort-relaxation (satisfaction) state.

In the above-mentioned evaluation of emotion using the six HEMM parameters, several emotional states were examined as examples. In the evaluation of more various emotional states, a specific emotional state may be evaluated by a selection combination of six parameters. The present disclosure provides a method that surpasses the evaluation limit of the conventional method of evaluating emotion only with a facial image. That is, the method according to the present disclosure obtains an HEMM synchronized with heart information in extracting micro-movement of the AU, and evaluates the emotion using this, thereby enabling more accurate emotional evaluation.

The system for performing the emotional evaluation as described above may include a computer system in which an imaging device is provided as described above. For this, a contact sensor for detecting ECG/PPG from a subject or a non-contact detection device based on an image may be provided.

The computer system includes a signal analyzer or signal processor based on hardware and software that analyzes the facial image and extracts micro-movements of the AU by specifying the AU based on the analysis result, and an evaluator or determination unit that evaluates or judges emotion using the signal analyzer or signal processor.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. An emotion evaluation method based on heart-face movement synchronization, the emotion evaluation method comprising:
taking a facial image of a subject using a camera;
extracting heart information of the subject using a heart information sensor;
extracting micro-movement information of one or more action units (AUs) defined in a face region from the face image by an image processor;
extracting heart-evoked micro-movement (HEMM) data from the micro-motion information in synchronization with a heartbeat characteristic signal obtained from the heart information by an information processor;
extracting one or more characteristic parameters for AU motion from the HEMM data; and
evaluating the emotion of the subject using the characteristic parameter by an emotion evaluator.

2. The emotion evaluation method of claim 1, wherein the heartbeat characteristic signal is obtained from PPG or ECG information.

3. The emotion evaluation method of claim 1, wherein the HEMM data is extracted from a peak power generation point in the heartbeat characteristic signal for a predetermined time (t).

4. The emotion evaluation method of claim 3, wherein the predetermined time (t) is set to 0.5 seconds.

5. The emotion evaluation method of claim 1, wherein at least one of Mean, standard deviation (SD), positive power peak (PPP), positive peak time (PPT), negative peak power (NPP), and negative peak time (NPT) from the HEMM data is calculated as the characteristic parameter.

6. The emotion evaluation method of claim 5, wherein the AU includes at least one of inner brow raise AU1, outer brow raise AU2, upper lid raise AUS, cheek raise AU6, lids tight AU7, nose wrinkle AU9, lip corner puller AU12, lip corner depressor AU15, lower lip depress AU16, lip stretch AU20, lip funneler AU23, and jaw drop AU26.

7. The emotion evaluation method of claim 1, wherein the AU includes at least one of inner brow raise AU1, outer brow raise AU2, upper lid raise AUS, cheek raise AU6, lids tight AU7, nose wrinkle AU9, lip corner puller AU12, lip corner depressor AU15, lower lip depress AU16, lip stretch AU20, lip funneler AU23, and jaw drop AU26.

8. The emotion evaluation method of claim 1, wherein the AU evaluates at least one of six basic emotions such as happiness HA, sadness SA, surprise SU, anger AN, disgust DI, and fear FE using the characteristic parameter.

9. The emotion evaluation method of claim 5, wherein the AU evaluates at least one of six basic emotions such as happiness HA, sadness SA, surprise SU, anger AN, disgust DI, and fear FE using the characteristic parameter.

10. The emotion evaluation method of claim 6, wherein the AU evaluates at least one of six basic emotions such as happiness HA, sadness SA, surprise SU, anger AN, disgust DI, and fear FE using the characteristic parameter.

11. An emotion evaluation system based on heart-face movement synchronization, the emotion evaluation system comprising:
facial imaging camera configured to photograph a subject's face;
a heart information extractor configured to extract heart information of the subject;
an information processor configured to process an image from the facial imaging camera to extract micro-movement data of an AU defined on the subject's face, wherein a micro-movement (HEMM) data of the AU is extracted based on the heart information of the subject, and a characteristic parameter for the movement of the AU is extracted from the micro-movement data; and
an emotion evaluator configured to evaluate an emotion displayed on the subject's face using the characteristic parameter.

12. The emotion evaluation system of claim 11, wherein the information processor extracts the HEMM data from a peak power generation point in the heartbeat characteristic signal for a predetermined time (t).

13. The emotion evaluation system of claim 11, wherein the information processor calculates at least one of Mean, standard deviation (SD), positive power peak (PPP), positive peak time (PPT), negative peak power (NPP), and negative peak time (NPT) from the HEMM data as the characteristic parameter.

14. The emotion evaluation system of claim 13, wherein the AU includes at least one of inner brow raise AU1, outer brow raise AU2, upper lid raise AUS, cheek raise AU6, lids tight AU7, nose wrinkle AU9, lip corner puller AU12, lip corner depressor AU15, lower lip depress AU16, lip stretch AU20, lip funneler AU23, and jaw drop AU26.

15. The emotion evaluation system of claim 14, wherein the AU evaluates at least one of six basic emotions such as happiness HA, sadness SA, surprise SU, anger AN, disgust DI, and fear FE using the characteristic parameter.

16. The emotion evaluation method of claim 2, wherein at least one of Mean, standard deviation (SD), positive power peak (PPP), positive peak time (PPT), negative peak power (NPP), and negative peak time (NPT) from the HEMM data is calculated as the characteristic parameter.

17. The emotion evaluation method of claim 3, wherein at least one of Mean, standard deviation (SD), positive power peak (PPP), positive peak time (PPT), negative peak power (NPP), and negative peak time (NPT) from the HEMM data is calculated as the characteristic parameter.

18. The emotion evaluation method of claim 4, wherein at least one of Mean, standard deviation (SD), positive power peak (PPP), positive peak time (PPT), negative peak power (NPP), and negative peak time (NPT) from the HEMM data is calculated as the characteristic parameter.
